(19) United States
(12) Patent Application Publication
(10) Pub. No.: US 2022/0249767 A1
(43) Pub. Date: Aug. 11, 2022

(54) METHOD FOR TREATMENT OF NEUROCOGNITIVE DECLINE

(71) Applicant: INVICTUS HEALTH, INC., Denver, CO (US)

(72) Inventors: Jeffrey I. Haugland, Denver, CO (US); Sam J. Peterson, Denver, CO (US)

(21) Appl. No.: 17/592,723

(22) Filed: Feb. 4, 2022

Related U.S. Application Data
(63) Continuation of application No. 17/029,417, filed on Sep. 23, 2020.
(60) Provisional application No. 63/010,825, filed on Apr. 16, 2020.

(51) Int. Cl. A61M 5/14; A61M 31/00; A61M 1/36; A61K 9/00; A61K 31/135; A61K 38/28; A61K 35/16; A61G 10/02

(52) U.S. Cl. CPC A61M 5/14; A61M 31/00; A61M 1/3693; A61K 9/0019; A61K 31/135; A61M 2210/0618; A61K 35/16; A61G 10/026; A61K 9/0043; A61M 2210/0693; A61M 2202/0415; A61K 38/28

(57) ABSTRACT

A method for treating a patient with a neurodegenerative disease including performing Ketamine Infusion Therapy on the patient, performing hyperbaric oxygen therapy on the patient, intranasally infusing plasma into the patient, and intranasally infusing diluted insulin into the patient. Performing hyperbaric oxygen therapy on the patient may include placing the patient into a hyperbaric chamber, sealing and pressurizing the hyperbaric chamber, and pumping oxygen into the hyperbaric chamber while it is pressurized. Before intranasally infusing plasma into the patient, the method may include drawing blood from the patient and centrifugally extracting the plasma from the drawn blood, such as platelet rich plasma (PRP). Before drawing blood from the patient, the method may include intravenously injecting the patient with supplements, such as a combination of magnesium, calcium, B-vitamins, Vitamin C, nicotinamide adenine dinucleotide (NAD+), and Glutathione.

### CROSS REFERENCE TO RELATED APPLICATION(S)

This application is a Continuation of U.S. application Ser. No. 17/029,417, filed Sep. 23, 2020 entitled “METHOD FOR TREATMENT OF TRAUMATIC BRAIN INJURIES”, which claimed the benefit of U.S. Provisional Patent Application Ser. No. 63/010,825, filed on Apr. 16, 2020, both of which are hereby incorporated by reference in its entirety for all intents and purposes.

### BACKGROUND OF THE INVENTION

### Field of the Invention

The present invention relates in general to medical treatments and procedures, and more particularly to a method for treatment of neurodegenerative diseases as well as traumatic brain injuries.

### Description of the Related Art

Traumatic Brain Injuries (TBIs) are a leading cause of death and disability in the United States. The Centers for Disease Control and Prevention (CDC) has estimated that approximately 1.5 million Americans survive a traumatic brain injury on an annual basis. The impact of TBI on society is immense, with TBI related healthcare expenditures eclipsing almost $50 billion dollars each year. However, the true burden of TBI is carried by the long-term complications suffered by its survivors and their loved ones who face progressive cognitive, motor and behavioral degradation due to neurodegenerative processes. The current standard of care for traumatic brain injuries centers around rehabilitation and pharmaceutical interventions that do not address the underlying causes of traumatic brain injuries. The lifetime costs of a patient's treatment for a traumatic brain injury are estimated at $85,000 to $3 million utilizing the current standard of care.

The conventional treatment protocol for traumatic brain injuries centers around symptom management. There are two primary methodologies of TBI symptom management, including pharmaceutical interventions and rehabilitation-based interventions. Pharmaceutical interventions include anti-anxiety medication to lessen feelings of nervousness and fear, anti-coagulants to prevent blood clots, anti-convulsants to prevent seizures, anti-depressants to treat symptoms of depression and mood instability, muscle relaxants to reduce muscle spasms, and stimulants to increase alertness and attention.

Rehabilitation-based interventions include physical therapy treatment to build physical strength, coordination, and flexibility, occupational therapy to help a person learn or relearn how to perform daily tasks (e.g., getting dressed, cooking, and bathing), speech therapy to work on the ability to form words and other communication skills, and training to learn how to use special communication devices if necessary. Rehabilitation-based interventions may also include the evaluation and treatment of swallowing disorders (dysphagia), psychological counseling to help a person learn coping skills, work on relationships, and improve general emotional well-being, vocational counseling which focuses on a person's ability to return to work, find appropriate opportunities, and deal with workplace challenges, and cognitive therapy including activities designed to improve memory, attention, perception, learning, planning, and judgment. For many people with TBI, cognitive therapy is among the most common types of rehabilitation.

Conventional treatment protocols serve only to regulate a patient's current state of being and teach patients to cope with their symptoms, yet do very little to address the underlying causes of a patient's symptoms.

### SUMMARY OF THE INVENTION

A method for treating a patient with a traumatic brain injury according to one embodiment of the present disclosure includes performing Ketamine Infusion Therapy on the patient, performing hyperbaric oxygen therapy on the patient, intranasally infusing plasma into the patient, and intranasally infusing diluted insulin into the patient. Performing hyperbaric oxygen therapy on the patient may include placing the patient into a hyperbaric chamber, sealing and pressurizing the hyperbaric chamber, and pumping oxygen into the hyperbaric chamber while it is pressurized. Before intranasally infusing plasma into the patient, the method may include drawing blood from the patient and centrifugally extracting the plasma from the drawn blood. Before drawing blood from the patient, the method may include intravenously injecting the patient with supplements.

A method for treating a patient with a traumatic brain injury according to another embodiment of the present disclosure includes treating the patient with Ketamine Infusion Therapy treating the patient with a first series of at least one series of hyperbaric oxygen therapy treatments after the treating the patient with Ketamine Infusion Therapy, drawing blood from the patient and extracting plasma from the drawn blood after each of the at least one series of hyperbaric oxygen therapy treatments, intranasally infusing the plasma into the patient after extracting plasma from the drawn blood, and intranasally infusing diluted insulin into the patient after intranasally infusing the plasma into the patient.

Treating the patient with Ketamine Infusion Therapy may include intravenously injecting one or more doses of Ketamine Hydrochloride. A first dosage may be approximately 0.5 milligrams per kilogram (mg/kg) of body weight, and each subsequent dosage may increase by approximately 0.2 mg/kg. In one embodiment, four doses of Ketamine Hydrochloride may be injected.

Each hyperbaric oxygen therapy treatment may include placing the patient into a hyperbaric chamber, sealing the hyperbaric chamber, pressurizing the hyperbaric chamber, and pumping oxygen into the hyperbaric chamber while it is pressurized. The patient may be kept in the hyperbaric chamber for at least one hour. The hyperbaric chamber may be pressurized between approximately 1.5 to 3 atmospheres absolute. The method may include pumping concentrated medical grade oxygen into the hyperbaric chamber while it is pressurized. Each of the at least one series of hyperbaric oxygen therapy treatments may include treating the patient with multiple hyperbaric oxygen therapy treatments at a rate of no more than one treatment per day over a period of 20 to 30 days.

The extracting plasma from the drawn blood may include centrifuging the drawn blood to extract platelet rich plasma (PRP). The method may include intravenously injecting the patient with supplements prior to or during drawing blood from the patient. The supplements may include intravenously a combination of magnesium, calcium, B-vitamins, Vitamin C, nicotinamide adenine dinucleotide (NAD+), and Glutathione.

Intranasally infusing diluted insulin into the patient may include intranasally infusing a solution of insulin diluted 4:1 with saline. The method may include intranasally infusing diluted insulin into the patient daily for a total of 20 infusions.

The method may include treating the patient with a second series hyperbaric oxygen therapy treatments after a first time of intranasally infusing the plasma into the patient, drawing the patient's blood again for a second batch of drawn blood and extracting a second batch of plasma from the second batch of drawn blood after the second series hyperbaric oxygen therapy treatments, intranasally infusing the second batch of plasma into the patient, and intranasally infusing a second batch of diluted insulin into the patient.

The method may further include treating the patient with a third series hyperbaric oxygen therapy treatments, drawing the patient's blood again for a third batch of drawn blood and extracting a third batch of plasma from the third batch of drawn blood after the third series hyperbaric oxygen therapy treatments, intranasally infusing the third batch of plasma into the patient, and intranasally infusing a third batch of diluted insulin into the patient. The third series hyperbaric oxygen therapy treatments may include no more than 5 hyperbaric oxygen therapy treatments.

### BRIEF DESCRIPTION OF THE DRAWINGS

The present invention is illustrated by way of example and is not limited by the accompanying figures, in which like references indicate similar elements. Elements in the figures are illustrated for simplicity and clarity and have not necessarily been drawn to scale.

FIG. 1 is a flowchart diagram of an initial consultation process for determining whether a patient is eligible for treatment in accordance with an embodiment of the present disclosure.

FIG. 2 is a flowchart diagram illustrating a neurodegenerative disease treatment process according to one embodiment of the present disclosure for patients that are considered candidates for the TBI treatment.

FIG. 3 is a flowchart diagram more specifically illustrating each of the set of treatments of FIG. 2 according to one embodiment of the present disclosure.

FIG. 4 is a flowchart diagram more specifically illustrating up to 3 sets of treatments that may be applied by substantially repeating the set of treatments of FIG. 3 according to one embodiment of the present disclosure.

### DETAILED DESCRIPTION

A novel neurodegenerative disease treatment protocol in accordance with that described herein utilizes existing medical imaging technology to accurately diagnose a patient and then delivers an effective treatment protocol. As compared to costly conventional treatments, which serve only to manage a patient's symptoms, a neurodegenerative disease treatment protocol in accordance with that described herein is designed to heal the brain at a cellular level to return a patient back to their previous quality of life faster and for lower cost.

Patients first undergo a series of quantitative tests to determine the scope and severity of their injuries. Patients then begin a treatment protocol of Ketamine Infusion Therapy, hyperbaric oxygen therapy, intranasal plasma therapy, and intranasal insulin infusion. This treatment protocol is designed to address traumatic brain injuries on a cellular level, reduce neuro-inflammation and deliver the biological resources necessary for tissue regeneration directly to the brain. A treatment protocol as described herein leverages the unique mechanisms of each individual treatment in order to deliver superior patient outcomes.

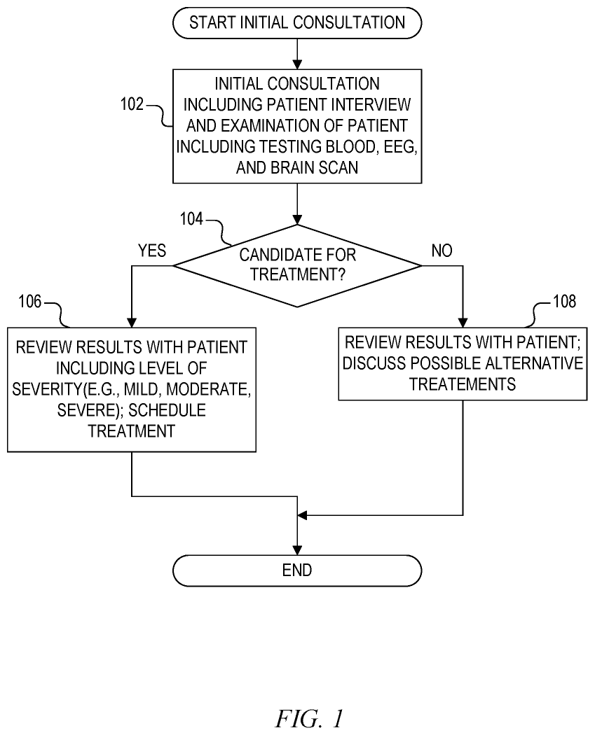

FIG. 1 is a flowchart diagram of an initial consultation process for determining whether a patient is eligible for treatment in accordance with an embodiment of the present disclosure. At a first block 102, an initial consultation is performed with the patient, which includes examination of the patient including conducting an interview, drawing blood, performing an electroencephalogram (EEG), and also performing a brain scan. The patient interview is performed to identify potential causes of and extent of brain injury, such as any combat experience including any possible blast-related injuries, accidents including automobile accidents or the like, any traumatic experiences that may have impacted the brain (e.g., blows to the head during a mugging or other injuries or abuses), hazardous activities including sports activities, boxing or kickboxing or the like, etc. The patient interview is also used to identify symptoms being experienced by the patient, such as headaches including migraines and corresponding frequency and severity, memory recall issues, sleep issues including insomnia, sleep apnea, etc.

The prospective patient's blood is tested and examined to identify hormone deficiencies, nutritional deficiencies, and inflammatory markers including the presence of cytokines or the like. A hormone deficiency is extremely common after a brain injury and could be a huge factor in some of the patient's symptoms. Nutritional deficiencies provide an idea of the patient's lifestyle, and cytokines are reviewed for possibly identifying a blood biomarker that positively identifies a brain injury.

The brain EEG tests a number of different measures, including physical reaction time, p300 delay (how long the brain takes to register an input), p300 voltage (total energy available for cognitive function/executive decision making), asymmetry (how well the left side communicates and works with the right side), and coherence (how efficiently the brain makes decisions). The brain EEG also measures the levels of alpha, beta, theta, and delta waves that may indicate why a patient experiences symptoms and that may provide information regarding how to relieve the symptoms more efficiently.

The brain scan, such as qSPECT or the like, provides brain imaging that may illustrate abnormally functioning of the brain. For example, the brain scan may identify areas of hypo-perfusion or “lack of blood flow” potentially caused by multiple traumatic brain injuries. The brain scan may also identify areas of hyper-perfusion or “increased blood flow,” in which blood flow may be increased to compensate for the areas of the brain that are struggling to function.

The brain scan of a particular patient diagnosed with TBI showed that at rest, overall cortical activity was reduced with a slightly decreased, patchy component. Focal areas of abnormal cortical hypoperfusion were noted in the bilateral orbitofrontal, bilateral anterior frontal, right lateral frontal, bilateral dorsolateral prefrontal, bilateral anterior and medial temporal, left posterior temporal, right superior parietal and right posterior cerebellar areas. Focal areas of abnormal subcortical hypoperfusion were noted in the left caudate areas. Focal areas of abnormally increased cortical perfusion were not noted. Cortical deactivation is noted with the concentration task. This particular patient would likely be considered eligible for TBI treatment in accordance with an embodiment of the present disclosure described herein.

At next block 104, the results of consultation, including results of the interview, blood tests, EEG, and brain scan, are reviewed and analyzed to determine whether the patient is a candidate for TBI treatment as described herein. If the patient is a candidate for TBI treatment, then at block 106 the results are reviewed with the patient including level of severity, TBI treatment is scheduled, and initial consultation is completed. The level of severity may be evaluated based on a general scale from mild to severe, which includes determination of intermediate moderate levels and very severe cases. The treatment schedule may be adjusted accordingly as further described herein. If the patient is not a candidate for TBI treatment, then at block 108 the results are reviewed with the patient along with any possible alternative treatments that may be available, and initial consultation is completed.

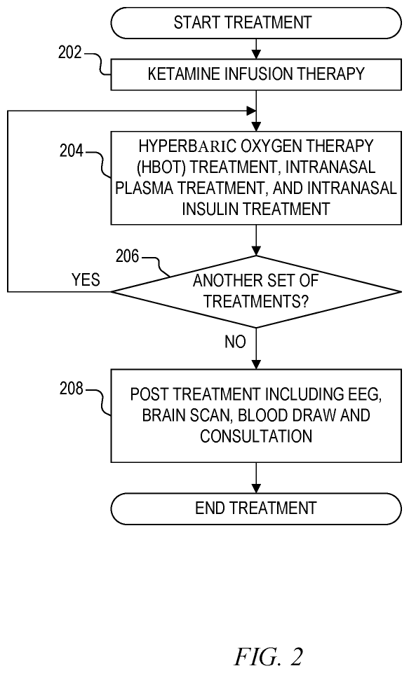

FIG. 2 is a flowchart diagram illustrating a treatment process according to one embodiment of the present disclosure for patients that are considered candidates for the treatment. At a first block 202, the patient undergoes Ketamine Infusion Therapy. Patients often suffer through the secondary phases of their injuries for years, enduring worsening symptoms and gradual cognitive and behavioral decline until finally seeking help. Often living with chronic pain and inflammation, these patients are also at high risk of developing comorbid depression. The Ketamine Infusion Therapy uses Ketamine Hydrochloride as the first treatment in the protocol to provide relief from depression or symptoms, downregulate inflammation, and upregulate brain derived neurotropic factor (BDNF) expression.

Ketamine Hydrochloride is a non-selective N-Methyl-d-aspartate (NMDA) glutamate receptor antagonist that elicits a dissociative psychedelic response in the brain and that may be used to treat Major Depressive Disorder (MDD). Traumatic brain injury patients are at a drastically increased risk of developing post-concussive anxiety and depression. Ketamine Hydrochloride is not only effective at relieving depression symptoms it is also anti-inflammatory in nature and promotes the release of BDNF into the cerebral spinal fluid of the patient following administration. The release of BDNF as well as a reduction in neuro-inflammation facilitate the growth of new neurons.

In one embodiment, Ketamine Hydrochloride is administered intravenously based on a patient's body weight at doses listed in the following Table I, measured in milligrams per kilogram of body weight (mg/kg):

TABLE I
Schedule of Ketamine Infusion Therapy Treatment and Dosage

| Treatment Number | DOSAGE |
|---|---|
| 1 | 0.5 mg/kg |
| 2 | 0.7 mg/kg |
| 3 | 0.9 mg/kg |
| 4 | 1.1 mg/kg |

To produce the greatest patient response to Ketamine, the TBI treatment protocol utilizes an increasing dosage schedule of Ketamine administered intravenously over four treatments administered over a period of about 2 weeks, such as, for example, 2 Ketamine treatments per week. Dosing begins at 0.5 mg/kg and increases, based on patient tolerance, by 0.2 mg/kg each subsequent infusion up to a maximum dose of 1.1 mg/kg at the fourth infusion.

The methodology for the gradually increasing the dosage schedule as described herein is based on research performed with other compounds, such as psilocybin, that suggests that the anti-depressant effects of psychedelic compounds are directly related to the novel experience that patients undergo while under the influence of the drug. The relief of depression symptoms can also be explained through the biochemical effects of the drug on the nervous system. It is noted that the dosage and number of treatments may be adjusted based on evaluation of effectiveness including patient tolerance.

BDNF is a vital neurotrophin that exhibits high levels of expression in mammalian brains. BDNF is responsible for the regulation of synaptic repair, protection, and growth, even in the presence of various neurotoxins and Reactive Oxygen Species (ROS). The idea that degenerative diseases of the nervous system may result from an insufficient supply of neurotrophic factors has generated great interest in BDNF as a potential therapeutic agent. Chronic inflammation plays a key role in the secondary injuries experienced by TBI patients. The immune response of macrophage cells to trauma is the release of proinflammatory cytokines, nitric oxide, and oxidative substance into the point of injury. Under normal conditions, the resulting upregulation of inflammation at the point of injury would signal the process of cellular healing to begin. However, in the case of microglial priming, microglial cells enter a state of prolonged activation in which they continually release proinflammatory cytokines.

Immediately following the completion of Ketamine Infusion Therapy, a set of treatments are administered at block 204 including a series of Hyperbaric Oxygen Therapy (HBOT) treatments, an intranasal plasma treatment, and an intranasal insulin treatment. Each of these therapies are further described herein below. At next block 206, it is queried whether the patient should undergo another set of treatments, and if so, the procedure loops back to block 204 in which another set of treatments are administered. The total number of treatments depends upon the level of severity of the neurodegenerative disease. Only one set of treatments may be administered for mild cases. A second set of treatments may be administered for moderate to mildly severe cases. A third set of treatments may be administered for severe to very severe cases. In one embodiment, the first and second sets of treatments are substantially the same, whereas the third set of treatments may be moderated or reduced as further described herein. It is noted that a portion of successive sets of treatments may overlap each other. For example, the intranasal insulin treatments may overlap subsequent HBOT treatments as further described herein.

Upon completion of the one or more sets of treatments at block 204 based on severity level, treatment advances to block 208 for post treatment. Post treatment may include EEG, brain scan, and blood draw in a similar manner previously described at block 102 for purposes of comparison and evaluation, and the treatment is completed.

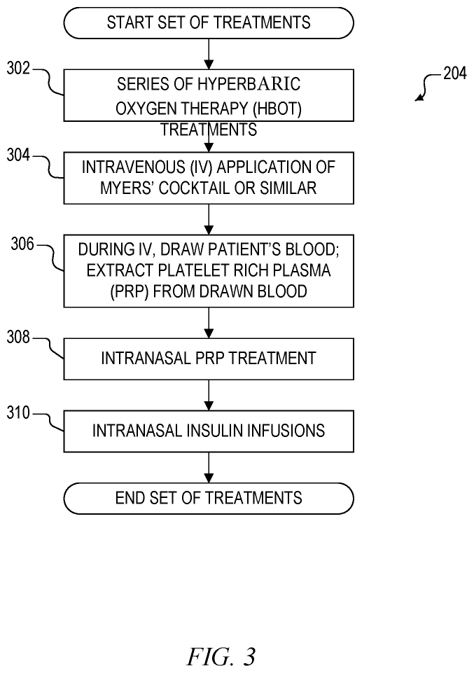

FIG. 3 is a flowchart diagram more specifically illustrating each of the set of treatments of block 204 according to one embodiment of the present disclosure. The procedure begins with a series of Hyperbaric Oxygen Therapy (HBOT) treatments at block 302. For each treatment, the patient enters or is placed into a hyperbaric chamber, which is then sealed. In one embodiment, the hyperbaric chamber is then pressurized between approximately 1.5 to 3 atmospheres absolute (ATA), which is between 7.5 to 15 pounds per square inch (PSI). Concentrated oxygen, preferably medical grade, may then be pumped into the hyperbaric chamber while it is pressurized. The patient remains in the hyperbaric chamber for about 60 to 90 minutes for each HBOT treatment session.

In one embodiment, the first series of HBOT treatments and also the second series, if applicable, may include 20 treatments each including one treatment per day over a time period of 20 to 30 days. In one embodiment, one HBOT treatment is performed per day for 20 days without skipping any days. In another embodiment, some days may be skipped as a matter of convenience. For example, 5 HBOT treatments per week may be performed without skipping more than one day between any two treatments. Ideally, the first 10 HBOT treatments should be performed in 10 days, one per day, without skipping any days between treatments. The third series of HBOT treatments, if applicable, may be substantially reduced to only about 2 treatments at an increased pressure, such as about 2-3 ATA depending upon the patient.

The increase in both pressure and oxygen concentration forces oxygen to dissolve into the bloodstream in much higher concentrations than at normal atmospheric pressure according to Henry's Law. Henry's Law, or S_{g}=kP_{g}, defines the relationship between pressure and solubility, in which S_{g} is solubility, k is Henry's Constant, and P_{g} is partial pressure. The sudden increase in blood/oxygen saturation signals the body to significantly upregulate the manufacturing of hemopoietic stem cells in the bone marrow. Once created, these stem cells are dumped into the bloodstream, in which 20 sessions of HBOT can increase levels of hemopoietic stem cells in the blood by a factor of eight (8×). As described further herein these stem cells are captured from the blood, concentrated, and then delivered directly to the brain via an intranasal infusion.

The brain is a highly aerobic organ. The brain receives about 15% of cardiac output, consumes about 20% of bioavailable oxygen, and utilizes about 25% of the total body glucose. At a standard healthy condition, at any given time, the brain is utilizing almost all oxygen/energy delivered to it. The regeneration process following a TBI uses a substantial amount of additional energy. The increased oxygen levels in the blood and body tissues produced by HBOT can supply the energy needed for brain repair. HBOT involves exposing the body to oxygen (e.g., 100% oxygen) while under greater than normal atmospheric pressures. HBOT harnesses the principle of Henry's Law to heal wounds. The patient enters the hyperbaric chamber and pressure is increased inside the chamber to between 1.5-3 ATA, which is approximately 1.5 to 3 times the normal atmospheric pressure at sea level. Once at pressure, medical-grade oxygen or the like is pumped into the chamber. The patient remains in the chamber breathing in concentrated oxygen for 60-90 minutes each treatment. The exposure to oxygen at pressure has profound effects on the body.

At sea level (which is 1 ATA, or about 14.7 psi) blood plasma oxygen concentration averages 0.3 ml per deciliter. Tissues at rest extract 5 to 6 milliliters (ml) of oxygen per deciliter of blood, assuming normal perfusion. Administering 100 percent oxygen at ambient pressure increases the amount of oxygen dissolved in the blood fivefold to about 1.5 ml per deciliter, and at 3 ATA, the dissolved-oxygen content is approximately 6 ml per deciliter, more than enough to meet resting cellular requirements without any contribution from oxygen bound to hemoglobin 32. Since the oxygen is dissolved into the plasma solution it can reach obstructed areas that red blood cells are unable to pass through. The hyperoxygenation resulting from HBOT also stimulates immune function by upregulating white blood cell creation, enhances cellular phagocytic capabilities, and accelerates neovascularization in hypoxic areas.

The primary mechanism of injury in a TBI is the diffuse shearing of axonal pathways and small blood vessels, also known as diffuse axonal injury. The follow-on secondary injuries caused by microglial priming; reactive oxygen species further impair the brain's ability to heal by creating inflammation-related tissue hypoxia. Global brain hypoperfusion, and its related tissue ischemia, detected in patients suffering from TBI, serves as a rate-limiting factor for any regenerative process. By increasing the oxygen level in blood and body tissues, HBOT can augment the body's natural repair mechanisms.

A concern regarding the delivery of additional oxygen into the body following a TBI is the creation of additional ROS in the body. Recent research has demonstrated that HBOT dramatically increases plasma oxygen saturation it also induces an upregulation in the body's antioxidant defenses. Experiments with invitro endothelial cells, the cells targeted for wound healing in HBOT therapies, found that HBOT activated the genetic expression of protective genes. Genes uncovered in this experiment include the 70-kilodalton heat shock protein (HSPA1A), heme oxygenase 1 (HMOX1), and metallothionein 1X (MT1X), which collectively can provide protection from metabolic, proteotoxic, and oxidative forms of stress 9. In the case of TBI, where ROS run rampant within hypoxic tissues, these effects provide protection from premature cellular apoptosis.

One of the most powerful second-order effects that HBOT has on the body is enhancing the creation of pluripotent bone marrow-derived stem/progenitor cells (SPCs). Pluripotent SPCs exhibit properties similar to embryonic stem cells and can differentiate into many different kinds of cells. After one HBOT treatment at 2.0 ATA for 2 hours the population of cells expressing the CD34+ stem cell marker doubled. Over the course of 20 HBOT treatments circulating 34D+ expressing cells increased eightfold. Further investigation into the relationship between the partial pressure of oxygen and the mobilization of SPC's has revealed that a correlation exists. The relationship between partial pressure and stem cell mobilization can be utilized therapeutically to treat TBI. Stem cells can be easily harvested along with hyper oxygenated plasma, concentrated and delivered to areas of the body or the brain that have been damaged by acute injury.

After the series of HBOT treatments, the procedure advances to block 304 in which an intravenous (IV) application of supplements are injected into the patient. The supplement may be according to a Myers' cocktail or a modified Myer's cocktail or the like. In one embodiment, the supplement includes magnesium, calcium, B-vitamins, Vitamin C, nicotinamide adenine dinucleotide (NAD+), and Glutathione. The IV application is administered to ensure hydration and electrolyte balance so that the patient's body is able to convert energy more efficiently. The IV application is administered for the first set of treatments and may be administered during the second and third set of treatments depending upon the patient.

Glutathione is a compound integral to cellular detoxification and is involved in the excretion of oxidative toxins from the body. Glutathione directly scavenges diverse oxidants: superoxide anion, hydroxyl radical, nitric oxide, and carbon radicals. Glutathione catalytically detoxifies hydroperoxides, peroxynitrites, and lipid peroxides. Another way glutathione protects cells from oxidants is through the recycling of vitamins C and E. Low levels of glutathione are associated with not only chronic exposure to chemical toxins but neurodegenerative disorders. To better facilitate the healing processes provided by PRP's inherent growth factors glutathione is delivered directly to the brain to neutralize ROS and other neurotoxins, paving the way for the regenerative effects for HBOT and PRP to take hold. By further combining the intranasally delivered PRP and Glutathione with NAD+, cellular metabolism can be further upregulated and cellular apoptosis can be averted.

NAD+ is a coenzyme found in all living cells. Substantial evidence has indicated that NAD+ plays a critical role in both cellular metabolism and apoptosis. Excessive poly (ADP-ribose) polymerase-1 (PARP-1) activation plays a significant role in ischemic brain damage. Increasing evidence has supported the hypothesis that PARP-1 induces cell death by depleting intracellular NAD+. By providing the cells of the brain an exogenous source of NAD+, it is desired to upregulate cellular metabolism and prevent further cellular apoptosis.

At next block 306 during the IV application, if applicable, the patient's blood is drawn and then the drawn blood is centrifuged to remove the red blood cells resulting in platelet rich plasma (PRP). PRP is an autologous concentration of human platelets derived from whole blood. Concentrations of human platelets are rich in the fundamental protein growth factors excreted by platelets to facilitate wound healing. These growth factors include the 3 isomeres of platelet-derived growth factor (PDGF), two of the numerous transforming growth factors (TGF1 and TGF2), vascular endothelial growth factor (VEGF), epithelial growth factor (EGF), insulin-like growth factor (IGF), fibroblast growth factor (FGF). Growth factors derived from PRP can contribute to tissue regeneration, by assisting cell migration, proliferation, differentiation, and extracellular matrix synthesis. PRP has been shown to accelerate healing following surgical procedures.

At next block 308, an intranasal PRP treatment is administered in which the PRP mixture developed at block 306 is infused into the patient's nasal cavity. In one embodiment, a nasal drug delivery device is filled with the PRP which is then injected into the patient's naval cavity via either or both nostrils. In one embodiment, up to 2 cubic centimeters (cc) may be injected into each nostril, although the particular dosage may vary from patient to patient. In one embodiment, the patient reclines on their back on a table or the like with their head tilted back so that their nostrils face upwards, and the PRP is injected into the patient's nostrils. In this manner, the plasma is able to bypass the blood brain barrier by following the olfactory nerves directly into the brain. The PRP growth factors enter the brain and adhere to areas of damage and inflammation, signaling to stem cells for immediate repair. Depending upon the severity of the TBI, the intranasal PRP infusions may be administered at up to 3 points during the TBI treatment, including once after each series of HBOT treatments. At least one goal of repeated PRP treatments is to repeatedly bathe the brain in growth factors and nutrients necessary to facilitate cellular healing. Any remaining PRP retrieved from the patient and not administered to the patient may be stored for subsequent treatments or after TBI treatment in the event any symptoms reoccur.

It is noted that intranasal delivery provides an expedient noninvasive method to deliver therapeutic agents to the brain. The blood-brain barrier (BBB) limits numerous therapeutic agents from entering the central nervous system (CNS) based on the molecular size or charge. Through intranasal administration therapeutic agents can bypass the BBB, ensuring delivery directly to the CNS in minutes. This is possible because of the unique connections that the olfactory and trigeminal nerves provide between the brain and the external environment. Intranasally administered therapeutics reach the CNS via the olfactory and trigeminal neural pathways. Both the olfactory and trigeminal nerves innervate the nasal cavity, providing a direct connection with the CNS. This method of administration is utilized to deliver a cocktail containing PRP and other vital nutrients directly to the brain to treat TBI.

After the intranasal PRP treatment at block 308, the procedure advances to block 310 for application of intranasal insulin infusions. For the intranasal insulin infusions, a diluted mixture of insulin is periodically injected intranasally using a nasal drug delivery device or the like, which is filled with the diluted insulin and intranasally injected into the nasal cavity via one or both of the patient's nostrils. In one embodiment, the insulin is diluted 4:1 with saline. In one embodiment, diluted mixture includes 8 cc saline and 2 cc insulin. In one embodiment, the intranasal insulin infusions at block 310 includes an infusion of diluted insulin once daily for a total of about 20 infusions during a treatment period of about 20 days. In one embodiment, the patient is taught how to dilute the insulin with saline and how to self-administer the intranasal insulin infusions. Each set of treatments is completed after completion of the intranasal insulin infusions.

It is noted that healing is a highly energy-intensive process. The compounded effects of neural inflammation and toxicity of TBI create an environment within the brain that is not conducive to healing. In order to assist and accelerate this process, the body is provided, via the intranasal insulin infusions, with more resources and help it to utilize them more effectively. Cellular respiration is the process by which cells convert biochemical energy, in this case, glucose and oxygen, into adenosine triphosphate (ATP). ATP is a complex organic chemical that provides the energy to drive many processes in living cells, including replication and cellular healing. By infusing diluted insulin intranasally, cells in the brain are able to convert the elevated levels of glucose and oxygen into ATP more efficiently, thereby providing them with the energy necessary for accelerated healing.

The inclusion of intranasal insulin into the treatment protocol is designed to create an additional synergistic effect with HBOT through the upregulation of cellular respiration. By simultaneously increasing the bioavailability of glucose through intranasal insulin and oxygen saturation through HBOT greater amounts of ATP can be produced and utilized to facilitate healing. Insulin is administered intranasally rather than intravenously to prevent the exacerbation of peripheral insulin resistance. Intranasal delivery provides a method for rapid delivery of insulin to the CNS along olfactory and trigeminal perivascular channels without adversely affecting blood insulin or glucose levels.

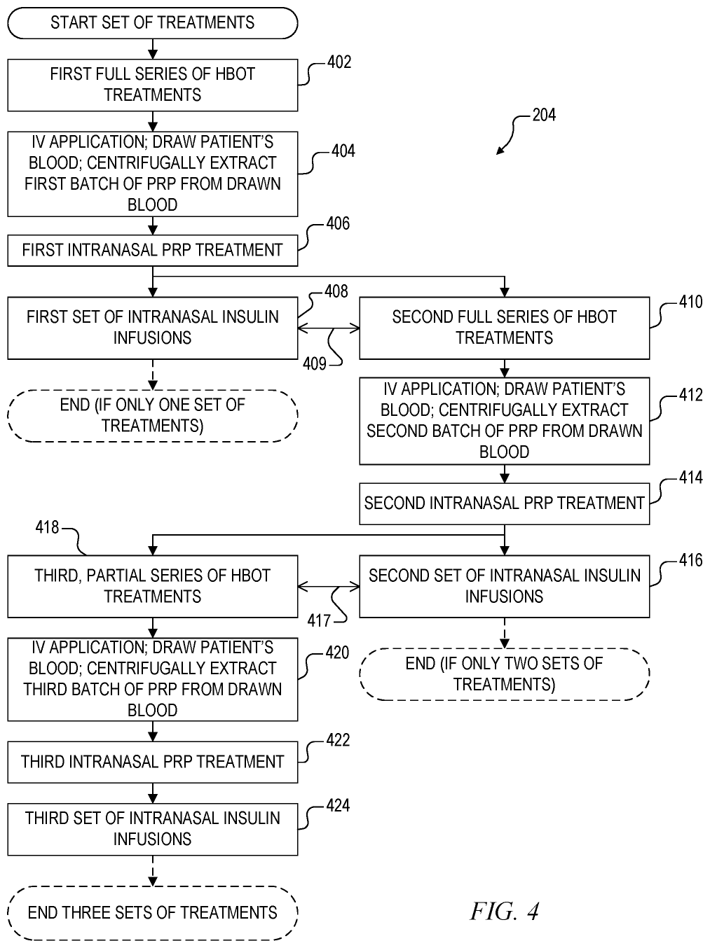

FIG. 4 is a flowchart diagram more specifically illustrating up to 3 sets of treatments that may be applied by substantially repeating block 204 according to one embodiment of the present disclosure. At block 402, a first full series of HBOT treatments are administered in the manner previously described for block 302, including, for example, 20 HBOT treatments over a period of 20 to 30 days. Next block 404 combines the procedures of blocks 304 and 306 previously described including IV application, patient blood draw, and extraction of a first batch of PRP from the drawn blood such as by centrifuging the drawn blood as previously described. At next block 406, the first intranasal PRP treatment is applied followed by the first round of intranasal insulin infusions at block 408. In the event the patient's condition or symptoms are mild, then the procedure may be completed after only one set of treatments.

If the patient's condition is more moderate in which another set of treatments is advised, then treatment may include a second set of treatments beginning at block 410 for a second full series of HBOT treatments. The second series of HBOT treatments at block 410 may be substantially the same as the first full set at block 402 including 20 HBOT treatments over a period of 20 to 30 days. Although the second series of HBOT treatments at block 410 may begin after completion of the first set of intranasal insulin infusions at block 408, the second series of HBOT treatments at block 410 may instead overlap the intranasal infusions at block 408 as indicated by arrow 409. For example, the first 6 to 7 intranasal insulin infusions at block 408 may be administered (or self-administered) during the same 20 to 30-day time period as the second series of HBOT treatments at block 410. This does not mean, of course, that any of the intranasal insulin infusions are administered while the patient is in the hyperbaric chamber during an HBOT treatment; instead intranasal insulin infusions may be administered according to the prescribed schedule between HBOT sessions.

After completion of both the first set of intranasal insulin infusions at block 408 and the second series of HBOT treatments at block 410, treatment proceeds to block 412 which is substantially the same as block 404 combining the procedures of blocks 304 and 306 previously described including IV application, patient blood draw, and extraction of a second batch of PRP from the drawn blood (such as by centrifuging the drawn blood as previously described). It is noted that depending upon the patient, the IV application may be omitted or modified such as a reduction of any one or more of the supplements administered during the IV. After the patient's blood is drawn and a second batch of PRP is extracted, the procedure advances to block 414 in which a second intranasal PRP treatment is applied followed by a second round of intranasal insulin infusions at block 416. In the event the patient's condition or symptoms are moderate and even in some mildly severe cases, the procedure is completed after two sets of treatments.

If the patient's condition is more severe in which another set of treatments is advised, then treatment may include a third set of treatments beginning at block 418 for potentially a third round of HBOT treatments. The third series of HBOT treatments at block 418, if administered, may be substantially reduced and include only 1 or 2 or more (possibly up to 5) HBOT treatments on a daily basis until completed. Although the third series of HBOT treatments at block 418 may begin after the second set of intranasal insulin infusions at block 416, the third series of HBOT treatments at block 418 may overlap the intranasal infusions at block 416 as indicated by arrow 417 in a similar manner as previously described. In this manner, the intranasal insulin infusions at block 416 may be administered (or self-administered) during the time period as the third series of HBOT treatments at block 418.

After completion of both the second set of intranasal insulin infusions at block 416 and the third series of HBOT treatments at block 418, treatment proceeds to block 420 which is substantially the same as block 404 combining the procedures of blocks 304 and 306 previously described including IV application, patient blood draw, and centrifugal extraction of a third batch of PRP from the drawn blood. It is noted that depending upon the patient, the IV application may be omitted or modified such as a reduction of any one or more of the supplements administered during the IV. After the patient's blood is drawn and a third batch of PRP is extracted, the procedure advances to block 422 in which a third intranasal PRP treatment is applied followed by a third round of intranasal insulin infusions at block 424. The procedure may be completed after three sets of treatments.

It is noted that the batches of PRP extracted from the drawn blood during blocks 404 and/or 412 may be used instead for the third intranasal PRP treatment, and that any PRP extracted from the patient's blood during the entire process may be saved for later use, such as in the event that neurodegenerative disease symptoms subsequently reoccur.

A treatment protocol in accordance with that described herein provides better patient outcomes by addressing the root cause of the symptoms from neurodegenerative disease at the cellular level. Life limiting symptoms can be effectively eliminated, the long-term risks of neurodegenerative disease in patients can be reduced or possibly eliminated, and direct and indirect healthcare costs associated with such disease may be significantly reduced. A treatment process in accordance with that described herein provides greater quality of life improvements at a lower overall cost. Due to the healing at a cellular level the protocol achieves, it also has the potential to treat patients suffering from Alzheimer's, Dementia, Parkinson's and chronic traumatic encephalopathy.

The present description has been presented to enable one of ordinary skill in the art to make and use the present invention as provided within the context of particular applications and corresponding requirements. The present invention is not intended, however, to be limited to the particular embodiments shown and described herein, but is to be accorded the widest scope consistent with the principles and novel features herein disclosed. Many other versions and variations are possible and contemplated. Those skilled in the art should appreciate that they can readily use the disclosed conception and specific embodiments as a basis for designing or modifying other structures for providing the same purposes of the present invention without departing from the spirit and scope of the invention.

1. A method for treating a patient with a neurodegenerative disease, comprising:
treating the patient with Ketamine Infusion Therapy;
after the treating the patient with Ketamine Infusion Therapy, treating the patient with a first series of at least one series of hyperbaric oxygen therapy treatments;
after each of the at least one series of hyperbaric oxygen therapy treatments, drawing blood from the patient and extracting plasma from the drawn blood;
after extracting plasma from the drawn blood, intranasally infusing the plasma into the patient; and
after intranasally infusing the plasma into the patient, intranasally infusing diluted insulin into the patient.

2. The method of claim 1, wherein the treating the patient with Ketamine Infusion Therapy comprises intravenously injecting at least one dose of Ketamine Hydrochloride.

3. The method of claim 2, wherein a first dosage is approximately 0.5 milligrams per kilogram (mg/kg) of body weight, and wherein each subsequent dosage increases by approximately 0.2 mg/kg.

4. The method of claim 2, wherein the intravenously injecting at least one dose of Ketamine Hydrochloride comprises intravenously injecting four doses of Ketamine Hydrochloride.

5. The method of claim 1, wherein the treating the patient with at least one series of hyperbaric oxygen therapy treatments comprises:
placing the patient into a hyperbaric chamber;
sealing the hyperbaric chamber;
pressurizing the hyperbaric chamber; and
pumping oxygen into the hyperbaric chamber while it is pressurized.

6. The method of claim 5, further comprising keeping the patient in the pressurized hyperbaric chamber for at least one hour.

7. The method of claim 5, wherein the pressurizing the hyperbaric chamber comprises pressurizing the hyperbaric chamber between approximately 1.5 to 3 atmospheres absolute.

8. The method of claim 5, wherein the pumping oxygen into the hyperbaric chamber while it is pressurized comprises pumping concentrated medical grade oxygen into the hyperbaric chamber while it is pressurized.

9. The method of claim 1, wherein each of the at least one series of hyperbaric oxygen therapy treatments comprises treating the patient with a plurality of hyperbaric oxygen therapy treatments at a rate of no more than one treatment per day over a period of 20 to 30 days.

10. The method of claim 1, wherein the extracting plasma from the drawn blood comprises centrifuging the drawn blood to extract platelet rich plasma (PRP).

11. The method of claim 10, further comprising intravenously injecting the patient with supplements prior to or during drawing blood from the patient.

12. The method of claim 11, wherein the intravenously injecting the patient with supplements comprises intravenously injecting the patient with a combination of magnesium, calcium, B-vitamins, Vitamin C, nicotinamide adenine dinucleotide (NAD+), and Glutathione.

13. The method of claim 1, wherein the intranasally infusing diluted insulin into the patient comprises intranasally infusing a solution of insulin diluted 4:1 with saline.

14. The method of claim 1, wherein the intranasally infusing diluted insulin into the patient comprises intranasally infusing diluted insulin into the patient daily for a total of 20 infusions.

15. The method of claim 1, wherein after a first time of intranasally infusing the plasma into the patient, further comprising:
treating the patient with a second series hyperbaric oxygen therapy treatments;
after the second series hyperbaric oxygen therapy treatments, drawing the patient's blood again for a second batch of drawn blood and extracting a second batch of plasma from the second batch of drawn blood;
intranasally infusing the second batch of plasma into the patient; and
intranasally infusing a second batch of diluted insulin into the patient.

16. The method of claim 1, wherein after a second time of intranasally infusing the plasma into the patient, further comprising:
treating the patient with a third series hyperbaric oxygen therapy treatments;
after the third series hyperbaric oxygen therapy treatments, drawing the patient's blood again for a third batch of drawn blood and extracting a third batch of plasma from the third batch of drawn blood;
intranasally infusing the third batch of plasma into the patient; and
intranasally infusing a third batch of diluted insulin into the patient.

17. The method of claim 16, wherein the treating the patient with a third series hyperbaric oxygen therapy treatments comprises treating the patient with no more than 5 hyperbaric oxygen therapy treatments.

18. A method of treating a patient with a neurodegenerative disease, comprising:
performing Ketamine Infusion Therapy on the patient;
performing hyperbaric oxygen therapy on the patient;
intranasally infusing plasma into the patient; and
intranasally infusing diluted insulin into the patient.

19. The method of claim 18, wherein the performing hyperbaric oxygen therapy on the patient comprises:
placing the patient into a hyperbaric chamber;
sealing and pressurizing the hyperbaric chamber; and
pumping oxygen into the hyperbaric chamber while it is pressurized.

20. The method of claim 18, prior to the intranasally infusing plasma into the patient, further comprising drawing blood from the patient and centrifugally extracting the plasma from the drawn blood.

21. The method of claim 20, prior to the drawing blood from the patient, further comprising intravenously injecting the patient with supplements.
